(19) United States
(12) Patent Application Publication
(10) Pub. No.: US 2022/0299525 A1
(43) Pub. Date: Sep. 22, 2022

(54) COMPUTATIONAL SENSING WITH A MULTIPLEXED FLOW ASSAYS FOR HIGH-SENSITIVITY ANALYTE QUANTIFICATION

(71) Applicant: THE REGENTS OF THE UNIVERSITY OF CALIFORNIA, Oakland, CA (US)

(72) Inventors: Aydogan Ozcan, Los Angeles, CA (US); Hyou-Arm Joung, Los Angeles, CA (US); Zachary S. Ballard, Los Angeles, CA (US); Omai Garner, Los Angeles, CA (US); Dino Di Carlo, Los Angeles, CA (US); Artem Goncharov, Los Angeles, CA (US)

(73) Assignee: THE REGENTS OF THE UNIVERSITY OF CALIFORNIA, Oakland, CA (US)

(21) Appl. No.: 17/612,575

(22) PCT Filed: May 22, 2020
(86) PCT No.: PCT/US2020/034349

§ 371 Date: Nov. 19, 2021

Related U.S. Application Data
(60) Provisional application No. 62/852,397, filed on May 24, 2019.

(51) Int. Cl. G01N 33/68; G01N 33/543; G06T 7/00

(52) U.S. Cl. CPC G01N 33/6893; G01N 33/54387; G06T 7/0012; G01N 2333/4737; G01N 2800/32; G06T 2207/20084; G06T 2207/10064

(57) ABSTRACT

A system for detecting the presence of and/or quantifying the amount or concentration of one or more analytes in a sample includes a flow assay cartridge having a multiplexed sensing membrane that has immunoreaction or biological reaction spots of varying conditions spatially arranged across the surface of the membrane defining an optimized spot map. A reader device is provided that uses a camera to image the multiplexed sensing membrane. Image processing software obtains normalized pixel intensity values of the plurality of immunoreaction or biological reaction spots and which are used as inputs to one or more trained neural networks configured to generate one or more outputs that: (i) quantify the amount or concentration of the one or more analytes in the sample; and/or (ii) indicate the presence of the one or more analytes in the sample; and/or (ii) determines a diagnostic decision or classification of the sample.

### RELATED APPLICATION

This Application claims priority to U.S. Provisional Patent Application No. 62/852,397 filed on May 24, 2019, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number 1648451, awarded by the National Science Foundation. The government has certain rights in the invention.

### TECHNICAL FIELD

The technical field generally relates to machine learning-based system that is used to read immunoreaction spots of a vertical flow assay (VFA) or a lateral flow assay (LFA) and determine optical configurations for the VFA/LFA and infer the target analyte concentration. In particular, the technical field relates to a system or platform that uses the machine learning-based framework to determine an optimal configuration of immunoreaction spots sensitive to C-Reactive Protein (CRP) (or other analyte or target) and conditions, spatially-multiplexed on a paper-based sensing membrane of the VFA/LFA which is also used to infer the target analyte concentration using the signals of the optimal VFA/LFA configuration.

### BACKGROUND

Computation and machine learning have great potential for improving diagnostics. By identifying complex and nonlinear patterns from noisy inputs, computational tools present an opportunity for automated and robust inference of medical data. For example, several studies have shown deep learning as a method to automatically identify tumors from an image, potentially enabling diagnostics in low-resource settings that lack a trained diagnostician. Additionally, computational solutions have been demonstrated earlier n the diagnostics pipeline to virtually stain pathology slides and enhance image resolution through the use of convolutional neural networks. Though much of this recent success is within the field of imaging, diagnostics that rely on biosensing can similarly leverage computational tools to improve sensing results and design future systems.

Point-of-care (POC) testing can especially benefit from computational sensing approaches. Due to their low-cost materials, compact designs, and requirement for rapid and user-friendly operation. POC tests are, unfortunately, often less accurate when compared to traditional laboratory tests and assays. For example, paper-based immuno-assays such as rapid diagnostic tests (RDTs) offer an affordable and user-friendly class of POC tests which have been developed for malaria, HIV-1/2, and cancer screening, among other uses. However, these RDTs lack the sensitivity and specificity needed for certain diagnostic applications largely due to issues of reagent stability, fabrication and operational variability, as well as matrix effects present in complex samples such as blood. Additionally, a well-known competitive binding phenomenon called the hook-effect can lead to false reporting of results, specifically in instances where the sensing analyte can be present over a large dynamic range. The hook-effect is a well-known problem with certain immunoassays whereby excess antigen or analyte will competitively bind with capture and/or detection antibodies giving a lower readout signal than is actually present. The hook-effect can also occur when blocking antibodies interfere with detection antibodies and results in a reduced signal. Therefore, computational tools alongside portable and cost-effective assay readers present a unique opportunity to compensate for some of these constraints. By quantifying the signals generated on paper-based substrates, machine learning algorithms have the potential to significantly improve the performance of POC sensors, without a significant hardware cost or increased complexity to the assay protocol.

### SUMMARY

In one embodiment, a computational paper-based flow assay cartridge is disclosed for cost-effective high-sensitivity C-Reactive Protein (hsCRP) testing, also referred to as cardiac CRP testing (cCRP) The flow assay cartridge may include both a vertical flow assay (VFA) cartridge as well as a lateral flow assay (LFA) cartridge. This low-cost and rapid (<12 min) flow assay cartridge uses a multiplexed sensing membrane and diagnostic algorithm based on neural networks to accurately quantify CRP concentration in the high-sensitivity range (i.e., 0-10 mg/L), as well as to identify samples outside of this range despite the presence of the hook-effect. While a CRP-based assay is disclosed herein, it should be appreciated that the flow assay cartridge may be used to detect or quantify the amount or concentration of other analytes in a sample. The analytes may include organic or inorganic molecules, compounds, or chemical species. The invention has particular application for biomolecules but the invention may also be used with other non-biological samples (e.g., environmental samples).

CRP is a general biomarker of inflammation, however slightly elevated CRP levels in blood can be an indicator of atherosclerosis, and have been shown to be a predictor for heart attacks, stroke, and sudden cardiac death for patients with and without a history of cardiovascular disease (CVD). Therefore, the hsCRP test is a quantitative test commonly ordered by cardiologists to stratify certain patients into low, intermediate, and high risk groups for CVD based off of clinically defined cut-offs: below 1 mg/L is considered low risk, between 1 and 3 mg/L is intermediate risk, and above 3 mg/L is high-risk. As a result, the hsCRP test requires a high degree of accuracy and precision, especially around the clinical cut offs, putting it out-of-reach of traditional paper-based systems. Additionally, in the presence of infection, tissue injury, or other acute inflammatory events, CRP levels can rise nearly three orders of magnitude, making hsCRP testing with immuno- and nephelometric-assays vulnerable to the hook-effect. As a result, samples with greatly elevated CRP levels can be falsely reported as within the hsCRP range, and therefore wrongly interpreted for CVD risk stratification.

To address these existing challenges of POC hsCRP testing, a system for detecting presence of and/or quantifying the amount or concentration of one or more analytes (e.g., CRP) in a sample was developed. In one particular embodiment, the system is a computational VFA-based sensing system was developed to jointly develop the CRP quantification algorithm and the multiplexed sensing membrane configuration, computationally selecting the most robust subset of sensing channels with which one can accurately infer the CRP concentration. A clinical study was performed with 85 patient serum samples and >250 VFA tests created over multiple fabrication batches, and compared the sensor performance to an FDA-approved assay and nephelometric reader (Dimension Vista System, Siemens). Blind testing results yielded an average coefficient of variation (CV) of 11.2% and a coefficient of determination (R²) of 0.95 over an analytical measurement range of 0 mg/L to 10 mg/L.

The POC analyte sensing system/device described herein can provide a rapid and cost-effective means to obtain valuable diagnostic and prognostic information for CVD, expanding access to actionable health information, especially for at-risk populations that often go underserved. Generally, the results also highlight computational sensing as an emerging opportunity for iterative assay and sensor development. Given a training data set, machine learning-based feature selection algorithms can be implemented to determine the most robust sensing channels for a given multiplexed system such as protein micro-array, well-plate assay, or multi-channel fluidic device, among others. This can therefore lead to optimized and cost-effective implementations of multiplexed bio-sensing systems for future POC diagnostic applications.

In one embodiment, a method of detecting the presence of and/or quantifying the amount or concentration of one or more analytes in a sample using a flow assay cartridge (vertical or lateral) is disclosed. The flow assay cartridge includes a plurality of absorption layers including a multiplexed sensing membrane. The method includes the operations of providing the flow assay cartridge with the multiplexed sensing membrane. The multiplexed sensing membrane has a plurality of immunoreaction or biological reaction spots of varying conditions spatially arranged across the surface of the membrane defining a pre-defined spot map, wherein the pre-defined spot map is determined by machine learning-based optimization to identify spot location and spot condition associated with the particular analyte(s) to be tested. That is to say the spatial location(s) as well as spot conditions (e.g., concentrations and the like) for the multiplexed sensing membrane are optimized pursuant to a machine learning task executed by machine learning software. The result is that a certain subset of the total number of spots in the multiplexed sensing membrane are used as the input to the trained neural network. The subset of spots is arrived at by machine learning optimization to select the best combination of spots and conditions that can computationally determine the analyte concentration.

The assay is performed by inserting a sample and reagent mixture into the flow assay cartridge. This may optionally be preceded by adding a buffer solution to prepare the various membrane layers for the sample and reagents. Likewise, after the sample and reagent mixture have been added to the flow assay cartridge, non-specific bound chemical species may be optionally washed with a second buffer solution. After allowing the sample and reagent mixtures to react with the spots of the multiplexed sensing membrane for a period of time in an incubation step (e.g., several minutes), the multiplexed sensing membrane is then subject to an imaging operation. In one embodiment, this may involve the separation or opening of the flow assay cartridge to allow access to the multiplexed sensing membrane. In other embodiments, the multiplexed sensing membrane may be imaged without the need to separate or open the flow assay cartridge. The multiplexed sensing membrane is imaged with a reader device configured to illuminate and obtain an image (or multiple images) of the multiplexed sensing membrane. The reader device may include a reader device that incorporates as part of thereof a portable electronic device with camera functionality. For example, the camera of a mobile phone (e.g., Smartphone) may be used as part of the reader device to capture image(s) of the multiplexed sensing membrane.

The image that is obtained using the reader device is then subjected to image processing to obtain normalized pixel intensity values of the plurality of plurality of immunoreaction or biological reaction spots. Normalized pixel intensity values may be obtained by a segmentation operation used to identify spot locations. Average or mean pixel intensity values within the segmented regions may be calculated followed by a background subtraction operation to create normalized pixel intensity values. The normalized pixel intensity values are then input to one or more trained neural networks configured to generate one or more outputs that (i) quantify the amount or concentration of the one or more analytes in the sample, and/or (ii) indicate the presence of the one or more analytes in the sample, and/or (iii) determine a diagnostic decision or classification of the sample.

In another embodiment, a system for detecting the presence of and/or quantifying the amount or concentration of one or more analytes in a sample includes a flow assay cartridge having therein a multiplexed sensing membrane having a plurality of immunoreaction or biological reaction spots of varying conditions spatially arranged across the surface of the membrane defining a pre-defined spot map of spot locations and spot conditions established for the one or more analytes. The spot map is, as is described herein in on embodiment, determined by machine learning-based optimization to identify spot location and spot condition(s) associated with the one or more analytes. The system includes a reader device that, in one embodiment, includes a housing defining an interior and having connector adapted to receive a portion of the flow assay cartridge containing the multiplexed sensing membrane, the reader device containing one or more illumination sources located in the interior portion and configured to illuminate the multiplexed sensing membrane, the reader device further including a mounting region configured to receive a portable electronic device with camera functionality such as mobile phone. A portable electronic device having a camera is disposed on or in the mounting region of the reader device, the camera of the portable electronic device being aligned along an optical path to obtain one or more images of the illuminated multiplexed sensing membrane. The portable electronic device may include a mobile phone or a portable camera.

The system includes a computing device configured to execute image processing software to obtain normalized pixel intensity values of the plurality of immunoreaction or biological reaction spots and then use the normalized pixel intensity values (as inputs) to one or more trained neural networks configured to generate one or more outputs that: (i) quantify the amount or concentration of the one or more analytes in the sample; and/or (ii) indicate the presence of the one or more analytes in the sample; and/or (ii) determine a diagnostic decision or classification of the sample. In some embodiments, the mobile phone itself acts as the computing device and performs image processing and/or executes the trained neural network. In other embodiments, the computing device may include a separate computing device (e.g., personal computer, laptop, server, etc.) that may be located locally with the reader or remotely from the reader. Alternative embodiments substitute a mobile or portable camera for the mobile phone with camera functionality.

### BRIEF DESCRIPTION OF THE DRAWINGS

FIG. 1A illustrates a side view of the portable reader device along with a flow assay cartridge (bottom portion) that is secured to an opto-mechanical attachment of the portable reader device. The opto-mechanical attachment is illustrated being secured to a mobile phone having camera functionality.

FIG. 1B illustrates an exemplary flow assay cartridge (two-part vertical flow assay cartridge) that is used to perform a vertical flow immunodiagnostic assay. The cartridge includes a bottom or lower portion that holds the multiplexed sensing membrane having a plurality of immunoreaction or bioreaction spots or locations formed therein or thereon along with a plurality of absorption pads. The top portion, which receives a liquid sample (and buffer or other assay reagents or solutions) contains a plurality of discrete porous layers as part of the assay.

FIG. 1C illustrates a front facing view of a mobile phone device (e.g., Smartphone) connected to the portable reader device. The screen or display of the mobile phone device illustrates an image of the multiplexed sensing membrane that was obtained with the camera of the mobile phone.

FIG. 1D illustrates a portable electronic device that is arranged to obtain an image of the multiplexed sensing membrane of the lower/bottom cartridge portion. A lens is located in the portable reader device is used to focus/defocus the image of the multiplexed sensing membrane onto the camera of the portable electronic device. Also illustrated are the light sources (e.g., light emitting diodes (LEDs)) and optional diffuser.

FIG. 2A illustrates a perspective view of a cartridge with the top or upper portion being twisted relative to the bottom or lower portion illustrating the detachable nature of the cartridge portions. Rotation in a first direction secures the top or upper portion to the bottom or lower portion while rotation in a second, opposite direction is used to remove the top or upper portion from the bottom or lower portion of the cartridge.

FIG. 2B illustrates perspective views of a single cartridge that includes a bottom or lower portion and an upper or top portion. The various discrete porous layers contained in each cartridge section or portion is also illustrated.

FIG. 3. illustrates a cross-sectional diagram of the porous layers contained in the flow assay cartridge. The multiplexed sensing membrane is denoted by the dotted line contained on the top layer of the bottom cartridge.

FIG. 4 illustrates a computing device that executes image processing software including the trained neural network (or multiple neural networks).

FIG. 5 illustrates an exemplary lateral flow assay cartridge that includes a multiplexed sensing membrane that can be used with the reader device.

FIG. 6 illustrates an embodiment of the multiplexed sensing membrane and the seven spotting conditions implemented for the clinical testing with the computational flow assay platform (right). The algorithmically determined spot map of the multiplexed sensing membrane is illustrated on the left.

FIG. 7 illustrates the tiered neural network that was used to output CRP qualitative and quantitative results. The first trained neural network (left) was used for qualitative output (high risk, intermediate risk, low risk) while three downstream neural networks were used to output quantitative results (concentration).

FIG. 8 illustrates the features selected from the cross-validation analysis are extracted from a blind testing image (left) and input into the neural network-based processing which infers the final CRP concentration. The clinical cutoffs for stratifying patients in terms of cardiovascular disease (CVD) risk are shown on the right.

FIG. 9 illustrates the image processing operations performed by the image processing software on the images.

FIG. 10 illustrates raw data from the training data set of clinical samples. The background-subtracted pixel averages of the immunoreaction spots are plotted against the CRP concentration. Each data point represents the average of like-spots and plotted per spotting condition. The marker shading (dark/light) and shape represent the different reagent batch ID (RID) and the fabrication batch ID (FID), respectively.

FIG. 11A illustrates the spot selection process (using training data set of clinical samples (Nₜᵣₐᵢₙ=209)). A heat-map (top left) is generated by plotting the cost function J_{m,p} across the sensing membrane. The cross-validation performance, both MSLE and the coefficient of variation (R²), is then plotted against the number of spots selected based off of J_{m,p} (bottom). The optimal subset of spots (top right) is then selected based off the optimal quantification performance indicated by the solid red marker.

FIG. 11B illustrates the condition selection process. Conditions are ranked based off of an iterative elimination method (top left), and the cross-validation performance is plotted against the number of conditions input into the quantification network. The optimal subset of conditions (top right) is then selected based off the optimal quantification performance indicated by the solid marker (dot).

FIG. 11C illustrates the cross-validation results using the selected features, where the ground truth CRP concentration is plotted against the predicted CRP concentration. The marker color and shape represent the different reagent batch ID (RID) and the fabrication batch ID (FID), respectively.

FIG. 11D is the Bland-Altman plot of the same cross-validation results, where the dashed red lines represent the ±standard deviation of the measurement difference from the tested vertical flow assays.

FIG. 12A illustrates the ground truth CRP concentration plotted against the VFA predicted CRP concentration (left y-axis) from blindly tested clinical samples. The dotted line represents a perfect match (y=x) and the solid line represents the linear best fit. The confidence score is plotted (right y-axis) for the samples classified as acute. The marker shading and shape represent the different reagent batch ID (RID) and fabrication batch ID (FID), respectively.

FIG. 12B illustrates a graph of the blind testing results for the low and intermediate CVD risk regimes, where the dotted lines represent the clinical cutoffs at 1 and 3 mg/L.

FIGS. 13A-13B illustrates blind testing results of the clinical samples using a multi-variable regression (shown here for comparison). FIG. 13A shows the ground truth CRP concentration plotted against the predicted CRP concentration from blindly tested clinical samples. The marker shading and shape represent the different reagent batch ID (RID) and the fabrication batch ID (FID), respectively. FIG. 13B illustrates the blind testing results for the low and intermediate CVD risk regime, where the dotted lines represent the clinical cut-offs at 1 and 3 mg/L.

FIG. 14 illustrates the normalized raw signals of five different spotting conditions implemented into the vertical flow assay as the cartridges are activated with varying CRP concentrations, which were spiked into CRP-free serum. The following spotting conditions were used within PBS buffer: 1) Primary CRP antibody (Ab) at 1 mg/mL; 2) the CRP antigen itself (Ag) at 2.1 mg/mL; 3) A mixture of the CPR Ab and Ag at 0.8 and 0.08 mg/L, respectively; 4) A mixture of the CPR Ab and Ag at 0.8 and 0.24 mg/mL; and 5) the CRP secondary Ab at 0.2 mg/mL.

### DETAILED DESCRIPTION OF THE ILLUSTRATED EMBODIMENTS

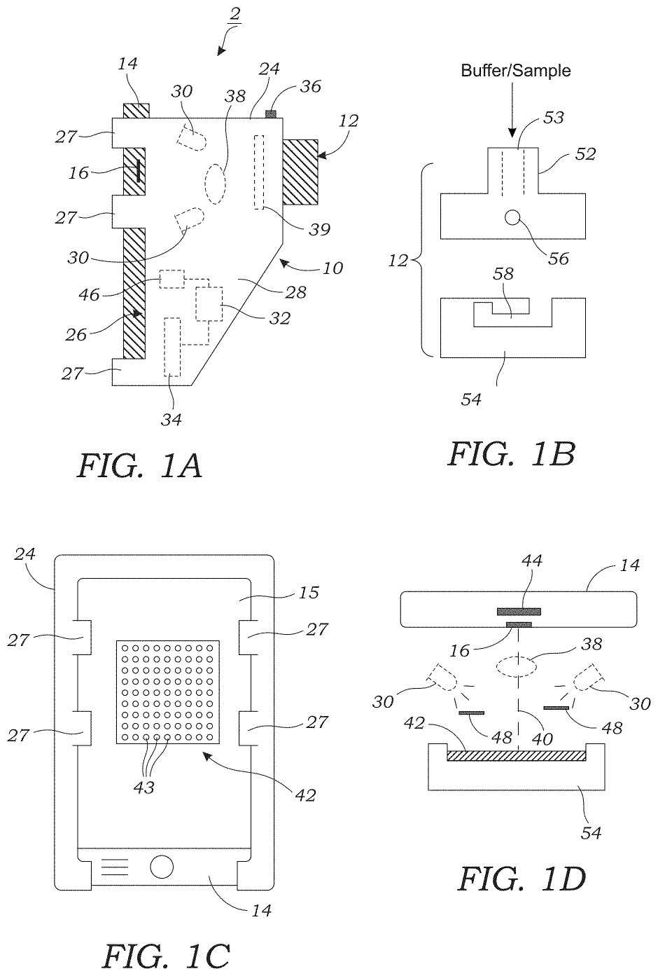

FIG. 1A illustrates a side view of a system 2 for detecting the presence of and/or quantifying the amount or concentration of one or more analytes in a sample according to one embodiment. The system 2 includes a reader device 10, a flow assay cartridge 12, a portable electronic device 14 having a camera 16 therein, and a computing device 18 (seen in FIG. FIG. 1E) that executes image processing software 20 including one or more trained neural network 22. In some embodiments, the portable electronic device 14 may be used as the computing device 18 which contains the image processing software 20 and trained neural network 22. In such case, there is no need for a separate computing device 18 as the computational resources of the portable electronic device 14 may be used. In other embodiments, however, the computing device 18 may include a separate device such as a personal computer, laptop, tablet PC, or remote server. In this embodiment, one or more images 50 obtained with the camera 16 are transferred from the portable electronic device 14 to the computing device 18. This transfer may be done using a conventional wired or wireless connection known to transfer data to/from portable electronic devices 14 known to those skilled in the art (e.g., Wi-Fi, Bluetooth, etc.). The computing device 18 may also reside within the reader device 10 itself.

The reader device 10 is preferably portable and/or hand-held in size an includes an opto-mechanical attachment 24 that is detachably mounted to a portable electronic device 14 having a camera 16 (see also FIG. 1D). The portable electronic device 14 may include a mobile phone or Smartphone such as that illustrated in FIGS. 1A, 1C, and 1D but may also include tablet PCs, a portable camera, microcomputers like the Raspberry Pi or the like, or other mobile computing platforms having embedded cameras. The opto-mechanical attachment 24 may include a mounting region 26 that is used to temporarily secure the portable electronic device 14 to the reader device 10. This mounting region 26 may include number of tabs, clips, fasteners 27, or a slot that is dimensioned to accommodate the portable electronic device 14 that enables the opto-mechanical attachment 24 to be removably secured to the portable electronic device 14. The opto-mechanical attachment 24 may be made from any number of materials including metals, polymers, plastics, and the like. In one preferred embodiment, the opto-mechanical attachment 24 may be formed using additive manufacturing techniques (e.g., 3D printing) although the invention is not so limited. The opto-mechanical attachment 24 forms a housing 28 that has an interior portion that contains one or more light sources 30 that are used to illuminate a multiplexed sensing membrane 42 which is described herein for obtaining image(s) 50 of the same. The one or more light sources 30 may include light emitting diodes (LEDs) (experiments described herein used 532 nm LEDs), laser diodes, or the like. The one or more light sources 30 are driven using driving circuitry 32 and powered by a power source 34 which may include one or more batteries. Alternatively, the power source 34 may be located external to the opto-mechanical attachment 24 and powered via a cord or the like (e.g., USB cord, power cord, or the like). A switch 36 located on the opto-mechanical attachment 24 may be used to turn the one or more light sources 30 on or off. In other embodiments, the switch 36 may be electronic and controlled using, for example, an application or app running on the portable electronic device 14.

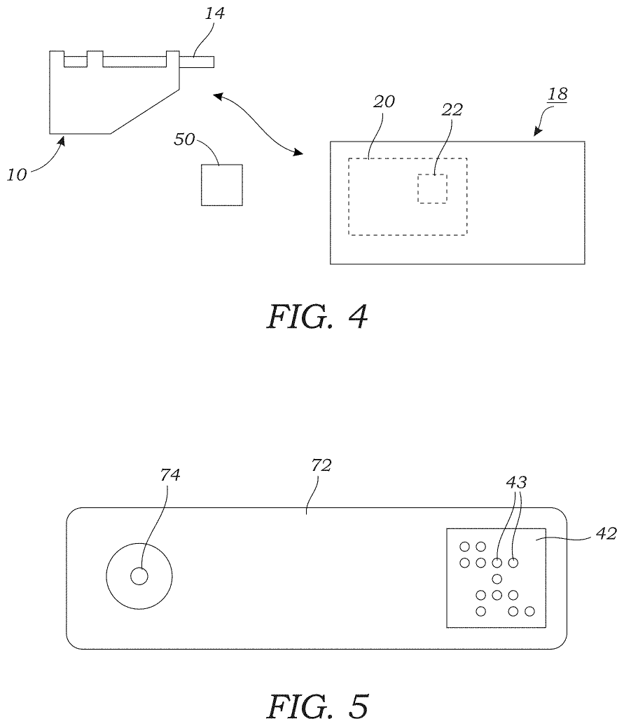

As seen in FIGS. 1A and 1D, the housing 28 of the opto-mechanical attachment 24 includes a lens 38 mounted therein that lies along an optical path 40 along with the lens (not shown) of the portable electronic device 14 to enable an in-focus field of view for imaging the multiplexed sensing membrane 42. FIG. 1D illustrates the optical path 40 formed between the multiplexed sensing membrane 42 and camera 16 of the portable electronic device 14. The camera 16 of the portable electronic device 14 includes an image sensor 44 (e.g., CMOS image sensors or the like) typically found in these types of devices for capturing images. The housing 28 may also include one or more processors 46 (e.g., microcontroller, application specific integrated circuits (ASICs)) that are used in conjunction with driving circuitry 32 to control operations of the reader device 10. In some embodiments, the one or more processors 46 may also perform various image processing and/or analysis functions described in more detail herein that are performed by image processing software 20. This may include, for example, image processing of the images 50 (FIGS. 4, 8, 9) obtained from the camera 16 of the portable electronic device 14 or even generating or outputting the results of the test (e.g., segmentation of images, background subtraction, normalization, or operating the trained deep neural network and generating output results).

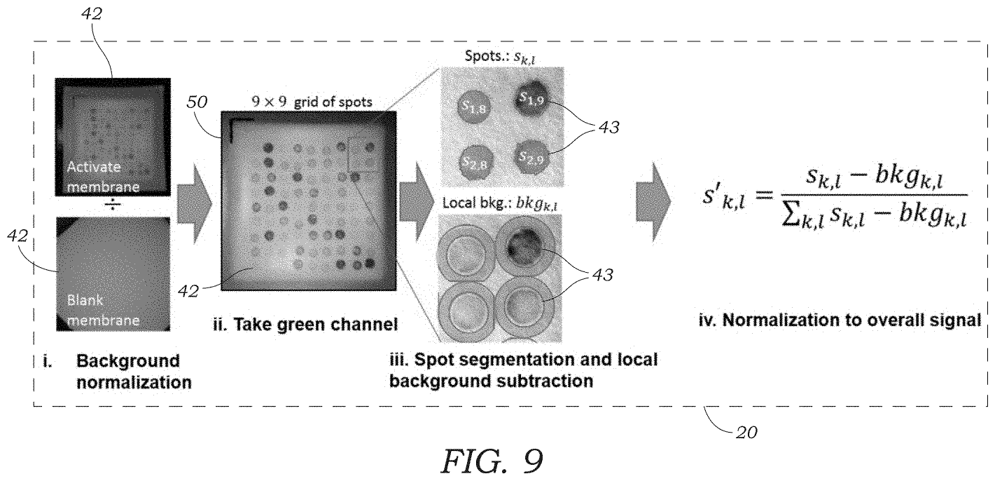

FIG. 9 illustrates the image processing operations performed by the image processing software 20 on the images 50. In operation (i) the image 50 of the multiplexed sensing membrane 42 is normalized to a universal blank background image (a blank sensing membrane with no spots 43), and in operation (ii) the green channel is taken or obtained (other color channels could also be used). Note that multiple images 50 may be obtained to increase detection sensitivity. In operation (iii) the spots are segmented through an automated algorithm and a local background is taken in a donut-shape outside of the segmented area. In operation (iv) the average pixel intensity of the local background bkg_{k,l} is subtracted from the average pixel intensity of the segmented spot s_{k,l} and normalized to the sum of all the background subtracted spot signals. Here, the indices k and l correspond to the row and column locations of the spots on the 9×9 grid, respectively.

The reader device 10 is configured to receive a portion of the flow assay cartridge 12 or all of the flow assay cartridge 12. This may include, for example, one part of a vertical flow assay cartridge 12. In other embodiments, the flow assay cartridge 12 is a unitary structure and cannot be separated and is secured to or inserted into the reader device 10 in its normal unopened state (see FIG. 5).

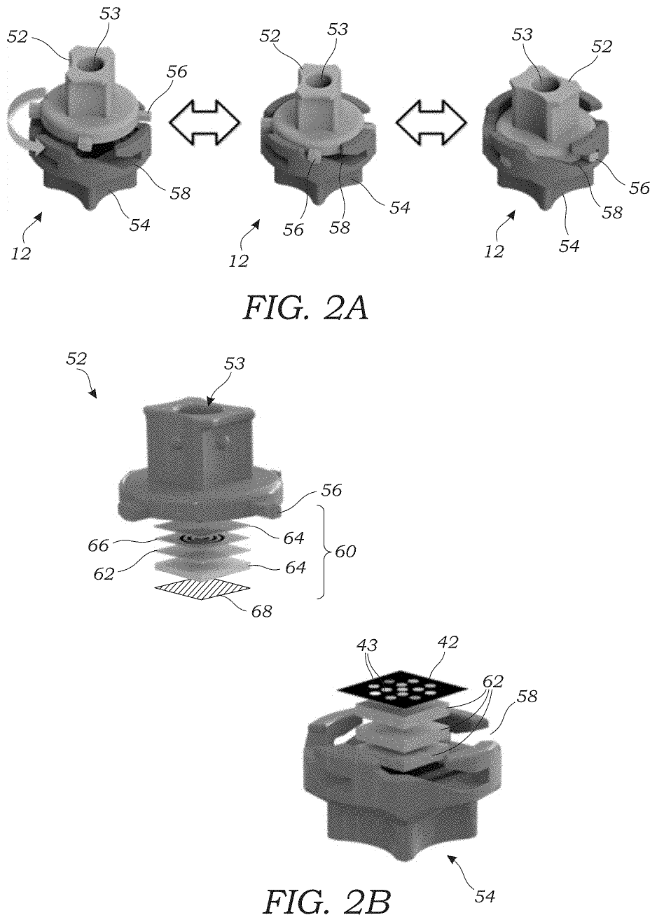

With reference to FIGS. 1B and 2A, the flow assay cartridge 12 includes a top or upper portion 52 that is detachably connected to a bottom or lower portion 54. The top or upper portion 52 may include one or more posts, detents, or bosses 56 that interface with a slot or recess 58 contained in the bottom or lower portion 54 of the flow assay cartridge 12. In this way, the top or upper portion 52 of the flow assay cartridge 12 is detachably connected to the lower portion 54 by twisting the upper portion 52 onto the lower or bottom portion 54 (FIG. 2A). The top or upper portion 52 includes an inlet 53 that receives the sample (and buffers or other reagents) as explained herein that flow through the flow assay cartridge 12 components.

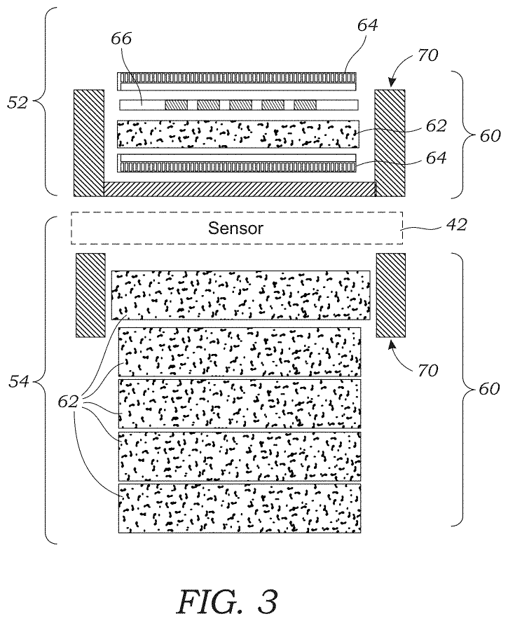

FIGS. 2B and 3 illustrates the components of the flow assay cartridge 12 contained in the top or upper portion 52, and the lower or bottom portion 54 of the flow assay cartridge 12. The top or upper portion 52 include an inlet 53 into which sample or other fluids (reagents, buffers, washes) are added for the vertical flow assay. A small volume of fluid (e.g., less than a few or several mL) is loaded into the inlet 53. The top or upper portion 52 and the lower or bottom portion 54 includes a stack of discrete porous layers 60 which are described in detail below. Some of the discrete porous layers 60 are used to absorb (e.g., absorption layers 62) fluid while other layers are designed to aid in fluid flowing particular directions. For example, the top or upper portion includes two asymmetric membranes 64. These asymmetric membranes 64 are asymmetric in that the pore size changes in the direction of the thickness of the membrane 64. The top such membrane 64 is oriented to place the side with the larger pores at the top while the bottom membrane 64 is oriented to place the side with the larger pores at the bottom. These asymmetric membranes 64 aid in lateral spreading (e.g., spreading layers). Some layers such as vertical flow diffuser layers 66 promote vertical (e.g., top-to-bottom) movement of fluid through the layer and inhibit lateral flow. Still other layers act as supporting structures (e.g., support layer 68) or support lateral flow (i.e., asymmetric membranes 64). The various layers may be held together and in place with an external or peripheral support 70 which in the experiments conducted herein was foam tape, although it should be understood that other materials and structures may be used. FIG. 3 illustrates a cross-sectional view of the various porous layers 60 contained in the top or upper portion 52 and the lower or bottom portion 54 of the flow assay cartridge 12 according to one embodiment. This sequence of porous layers 60 was used for the CRP-based vertical flow assay cartridge 12 tested herein. Table 1 below shows material specifications and cost per layer.

TABLE 1

| Layer | Material Specification | Cost (¢) |
|---|---|---|
| Asymmetric Membrane | Vivid GX, (1.2 × 1.2 cm) | 0.5 |
| (64) | | |
| Vertical Flow Diffuser | NC membrane, 0.45 μm | 2.9 |
| (66) | pore size (1.2 × 1.2 cm) | |
| Absorption Pad (62) | (1.2 × 1.2 × 0.1 cm) | 1.4 |
| Asymmetric Membrane | Vivid GX, (1.2 × 1.2 cm) | 0.5 |
| (64) | (reverse orientation) | |
| Supporting Membrane | NC membrane, 0.22 μm | 2.9 |
| (68) | pore size (1.7 × 1.7 cm) | |
| Multiplexed Sensing | NC membrane, 0.22 μm | 2.9 |
| Membrane (42) | pore size (1.7 × 1.7 cm) | |
| Absorption Pad (62) | (1.4 × 1.4 × 0.18 cm) | 1.4 |
| Absorption Pad (Stack) | (1.2 × 1.2 × 1.2 × | 5.6 |
| (62) | 0.18 cm) × 4 pads | |
| Foam Tape (70) | | 0.7 |
| | Total Cost | 18.8¢ |

Referring to FIGS. 1D, 2B and 3, the lower or bottom portion 54 of the flow assay cartridge 12 holds the multiplexed sensing membrane 42 with a plurality of spatially multiplexed immunoreaction spots or locations 43 formed therein. The multiplexed sensing membrane 42 may be made from nitrocellulose or other paper material. Each of the plurality of spatially multiplexed immunoreaction spots or locations 43 may include one more of a protein, antigen, antibody, nucleic acid, aptamer, or enzyme. In one particular embodiment, the spots or locations 43 are disease-specific antigens and/or antibodies. In this regard, the antigens and/or antibodies are biomarkers for a particular condition or disease state. As explained herein, for CRP testing, the spots or locations 43 included CRP capture antibodies (Ab), CRP antigen (Ag), combinations of these, and secondary CRP antibodies. Different concentrations of these may be used in the spots or locations 43. Different numbers of spots or locations 43 may be used depending on the assay but is typically more than three and less than around one-hundred spots. In the experiments herein, there were eighty-one (9×9 array) spots or locations 43. Some spots or locations 43 may also be used as fiducial marks to that can be used to register before and after images of the multiplexed sensing membrane 42. The spots or locations 43 that are formed on the sensing membrane 42 may be defined on a nitrocellulose membrane (e.g., 0.22 μm pore size) by a black wax-printed (or other hydrophobic material) barrier, where each spot or location 43 is pre-loaded with a different capture-antigen/antibody or antigen/antibody epitope-containing peptide to enable multiplexed sensing information within a single test. The spatially isolated immunoreaction spots or locations 43 are defined by wax printed barriers, allowing for different capture antigens to be spotted on the nitrocellulose sensing membrane 42. For example, after printing, the multiplexed sensing membrane 42 is incubated for 30 seconds at 120° C. in an oven to allow the printed wax to melt and diffuse downward into the nitro-cellulose. Each of the plurality of sensing spots 43 is then loaded with a small amount (e.g., ˜0.1 μL-several μL) of capture solution (containing antigen, antibody, protein, aptamer, etc.), and allowed to dry for 30 minutes at room temperature. Then, the sensing membrane 42 is dipped in 1% BSA in PBS solution for 30 minutes to block non-specific binding, and the sensing membranes 42 are again dried for 10 minutes at 37° C. in a dry oven. As seen in FIGS. 2B and 3, one or more adsorption layers 62 (e.g., a thick pad if formed from multiple layers) is located beneath the multiplexed sensing membrane 42 in the lower or bottom portion 54 of the flow assay cartridge 12.

To perform the assay with the system 2 a user sample is collected. The user sample may include, for example, a small (less than 1 mL) serum sample obtained from a human mammal. Other bodily fluids besides serum may also be tested (e.g., whole blood, saliva, semen, urine, sweat, and the like). The sample is then pre-processed, for example, by undergoing dilution. The flow assay cartridge 12 is assembled if not already done so. This includes securing the top or upper portion 52 to the lower or bottom portion 54. A small volume (e.g., 200 μL) of buffer is placed into the inlet 53. Gravity and the natural wicking motion move the fluid through the stack of porous layers 60. This operation may take several seconds (e.g., 30 seconds). Next, a small volume of the serum sample (e.g., 50 μL although it may be more or less) is mixed with an equal amount of gold-nanoparticle (Au NP) conjugate solution and the mixture is placed into the inlet 53 and allowed to absorb (i.e., gold nanoparticle conjugated to an antibody). Another small volume (e.g., 400 μL) of buffer is placed into the inlet 53 to wash away nonspecifically bound proteins and Au NPs. Gold nanoparticles conjugated with an antibody are bound to the immobilized analyte in a sandwich structure, resulting in a color signal that is generated at the spots or locations 43 of the multiplexed sensing membrane 42. The flow assay cartridge 12 is then allowed to incubate for several minutes (e.g., 10 minutes). After incubation, the top or upper portion 52 is removed from the lower or bottom portion 54 and the lower or bottom portion 54 that contains the multiplexed sensing membrane 42 is secured to the reader device 10 and imaged. This color signal response (e.g., pixel intensity) of the spots or locations 43 is captured in images 50 obtained using the reader device 10. The housing 28 may include a similar post, detent, or boss 56 that interfaces with the slot or recess 58 on the lower or bottom portion 54 of the flow assay cartridge 12 using a similar twisting engagement/disengagement as used with the upper or top portion 52 as described herein. The color signal response may include a single-color channel captured by the image sensor 44. For example, as described herein, the green channel is used to obtain pixel intensities at each spot or location 43.

While gold nanoparticles conjugated with an antibody was used in connection with the experiments described herein, it should be understood that other tags or reporters may be conjugated with an antibody. These include, by way of example, quantum dots conjugated to an antibody or antigen, or fluorescing reporter molecules or probes that emit fluorescence in response to excitation light. In the last instance, a filter interposed in the optical path 40 can filter out excitation light but allow transmission of emitted fluorescence from the spots 43 which can be imaged by the camera 16 (i.e., spectrally filtering the illumination source(s) and fluorescence signal(s)). In addition, it should be appreciated that the particular tag or reporter that is conjugated to a molecular probe, antibody (or multiple probes or antibodies) that is used in the assay workflow may itself be stored, in dry form, in one or more of the porous layers 60 (e.g., paper) of the cartridge 52. As liquid is flowed through the cartridge 52 (e.g., buffer and/or sample), these dry reagents are wetted and then can interact with the spots 43 of the multiplexed sensing membrane 42.

FIG. 1C illustrates a screen or display 15 of the portable electronic device 14 showing an obtained image of the multiplexed sensing membrane 42. Each individual spot or location 43 on the multiplexed sensing membrane 42 will have a particular color or intensity signal response that is subject to image processing using image processing software 20 and then input to the trained neural network 22 (FIG. 4) which is used to generate one or more outputs. The output(s) from the trained neural network 22 may: (i) quantify the amount or concentration of the one or more analytes in the sample, and/or (ii) indicate the presence of the one or more analytes in the sample, and/or (iii) determine a diagnostic decision or classification of the sample. This may include a qualitative output (e.g., low risk, medium risk, high risk, positive, negative) or the output may include a quantitative output (e.g., numerical concentration or level of analyte).

The portable electronic device 14 may include an application or “app” that is executed on the portable electronic device 14 that includes a graphical user interface (GUI) that can be used to run the assay or test and display results therefrom. For example, the GUI may display an image of the multiplexed sensing membrane 42 (either raw or after image processing) as well as the quantified intensity values of the spots or locations 43. The GUI may also display one or more of: patient ID, test location, test time, test type (e.g., CRP), diagnosis (e.g., positive (+), negative (−), low risk, intermediate risk, high risk), antigen/antibody concentration, cartridge ID, and the like.

FIG. 5 illustrates an embodiment of the flow assay cartridge 12 in the form of a lateral flow assay (LFA). The flow assay cartridge 12 includes a body or housing 72 an inlet 74 that receives the sample to be tested. The multiplexed sensing membrane 42 is also located in the flow assay cartridge 12 and includes spots 43 formed thereon or therein. The multiplexed sensing membrane 42 may be imaged by the reader device 10 as described herein. In this embodiment, however, the flow assay cartridge 12 does not have to be opened and instead can be inserted into the reader device 10 to place the multiplexed sensing membrane 42 in the optical path 40 for imaging. For example, the flow assay cartridge 12 can be inserted into the optional slot 39 in the opto-mechanical attachment 24.

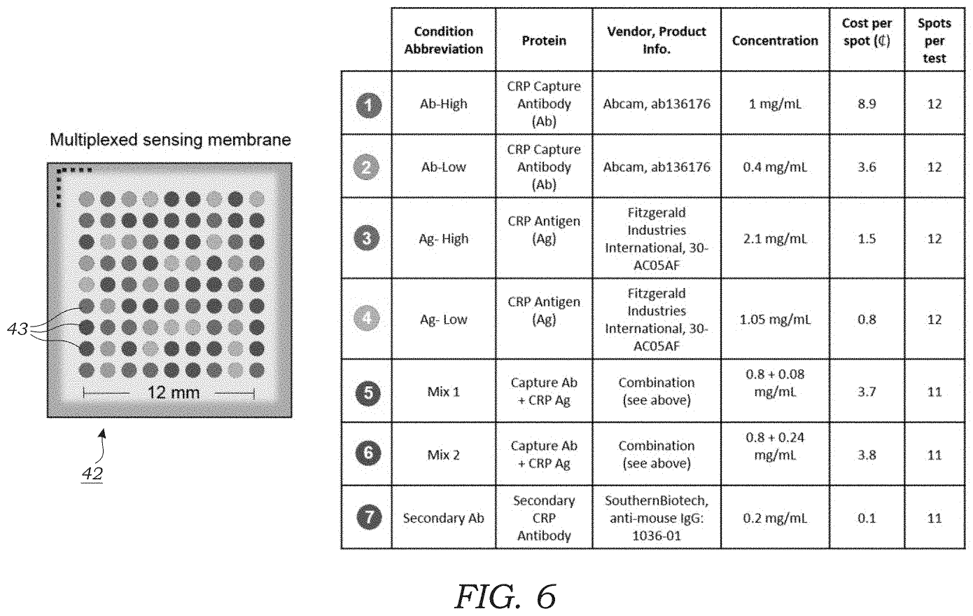

As explained herein, the actual spot map of spots 43 that is used to generate the output(s) (either qualitative or quantitative) is pre-determined. In one specific implementation or embodiment, this pre-determined map is determined by a machine learning-based optimization process to identify spot location(s) and/or spot condition(s) associated with the particular analyte(s) to be tested. FIG. 6 illustrates multiplexed sensing membrane 42 used in experiments described herein. The multiplexed sensing membrane 42 included a 9×9 array of spots 43 of various conditions (listed in adjacent table in FIG. 6).

The spatial location(s) of the spots 43 as well as the condition(s) of the spots 43 (e.g., concentrations and the like) for the multiplexed sensing membrane 42 are optimized pursuant to a machine learning task executed by machine learning software. The result is that a certain subset of the total number of spots in the multiplexed sensing membrane 42 are used as the input to the trained neural network 22. The subset of spots is arrived at by machine learning optimization to select the best combination of spots and conditions that can computationally determine the analyte concentration. The particular spot map for a particular test may be contained as part of test information that could be included for each test in the form of a Quick Response (QR) code, bar code, serial number, or other indicia associated with the flow assay cartridge 12 or could alternatively be logged into a GUI by the user before the measurement data are processed by the trained neural network 22. The spot map may also be used to manufacture or fabricate the actual particular spot map that is used in the multiplexed sensing membrane 42. In the later instances, fewer spots 43 need to be created which may save on reagent costs.

Thus, in one embodiment, after machine learning optimization has determined the particular spot map to be used for a particular analyte, the multiplexed sensing membrane 42 is then manufactured or fabricated only with those spots 43 that are in the map. For example, a 9×9 array of spots 43 (eighty-one total spots 43) may have initially been placed on the multiplexed sensing membrane 42 but after machine learning optimization a subset of these spots 43 were determined to be needed (e.g., thirty). Each of the spots 43 may be unique in some embodiments. In other embodiments, however, multiple spots 43 located in different spatial areas may be made of the same constituents (e.g., antigen, antibody, mixes thereof) to provide additional signals or data that is then used by the downstream trained neural network(s) 22. In another embodiment, the flow assay cartridge 12 may contain a multiplexed sensing membrane 42 fabricated or manufactured with the full array of spots 43 formed thereon. Machine learning optimization may then be performed at the site of use which is tailored to the particular application.

The image 50 that is obtained using the reader device is then subjected to image processing with image processing software 20 to obtain normalized pixel intensity values of the plurality of plurality of immunoreaction or biological reaction spots. Normalized pixel intensity values may be obtained by a segmentation operation used to identify spot locations. Average or mean pixel intensity values within the segmented regions may be calculated followed by a background subtraction operation to create normalized pixel intensity values. The normalized pixel intensity values are then input to one or more trained neural networks configured to generate (i) an output that quantifies the amount or concentration of the one or more analytes in the sample, (ii) indicates the presence of the one or more analytes in the sample, or (iii) determines a diagnostic decision or classification of the sample.

As explained herein, after loading and reaction of the sample and other reagent mixture in the flow assay cartridge 12, the multiplexed sensing membrane 42 is then imaged with the portable electronic device 14 that is secured to the reader device 10. The image(s) 50 that are obtained with the camera 16 are then subject to image processing using image processing software 20. This includes detection and segmentation of the spots 43. Following the detection and segmentation operation, the image processing software 20 then assigns a spot signal to each spot 43 as described by Eq. 1. The average pixel intensity of each spot 43 is calculated and subtracted by the pixel-averaged background followed by normalization to all spots 43 on the multiplexed sensing membrane 42.

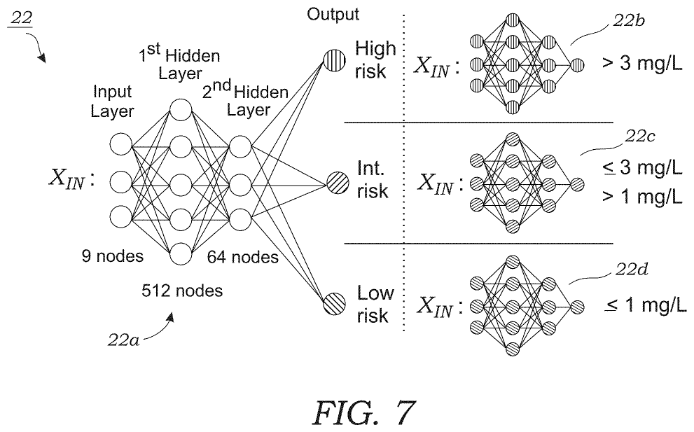

The spot signal for each spot 43 is then fed to a trained neural network 22. The trained neural network 22 used herein was a tiered neural network architecture as seen in FIG. 7 with a cost function of mean-squared logarithmic error (MSLE). As an alternative, a single neural network 22 with multiple hidden layers, in contrast to the tiered structure, could also be used in providing an accurate and generalizable model. FIG. 7 illustrates the trained neural network 22 with a tiered network structure used for cross-validation. The first tier 22a of the trained neural network classifies a given sample, defined by the input X_{IN}, into the high, intermediate, or low risk hsCRP regime based off of clinical cut-offs of 1 and 3 mg/L. The output of this first network 22a is a classification or qualitative output that classifies the sample as one or high risk, intermediate risk, or low risk. The second tier of the trained neural network 22 then uses three separate networks 22b, 22c, 22d trained with samples within each particular regime to quantify the CRP concentration of the sample. The output of these networks 22b, 22c, 22d is a quantitative output (e.g., concentration or concentration range of the sample). To avoid edge effects, each quantification network 22b, 22c, 22d is trained with samples within their cut-off as well as with samples within ±50% of the corresponding cut-off value. Each layer was trained with 50% dropout, ReLu (Rectified Linear Unit) activation function, and a batch size of 22, as determined via a hyper-parameter search. For simplicity, every neural network 22b, 22c, 22d used the same architecture and hyper-parameters, differing only in the output layer (i.e. classification or quantification) and the training data.

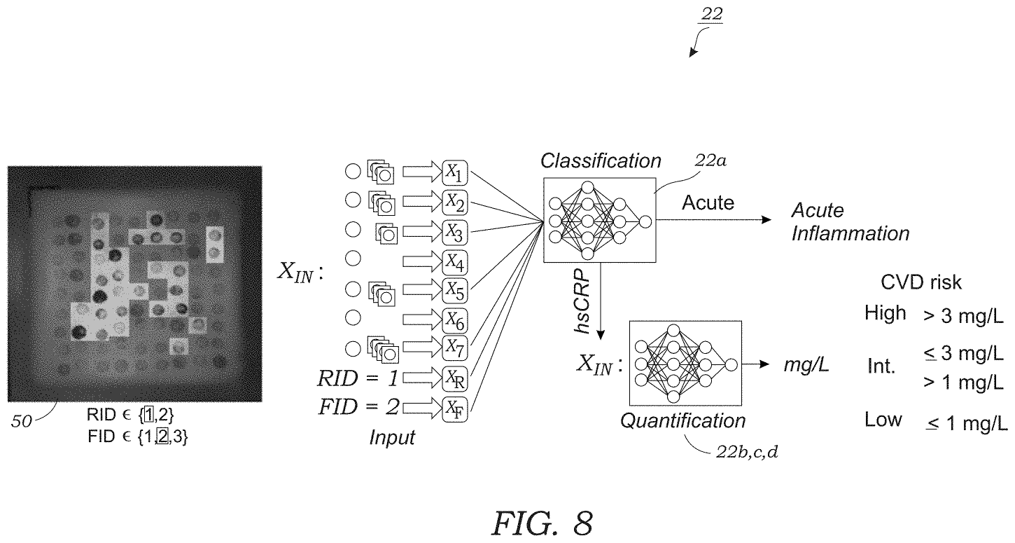

FIG. 8 illustrates an image 50 of the multiplexed sensing membrane 42 as well as a schematic of the trained neural network 22. The image 50 illustrates the stop map being used as the input to the trained neural network 22. In this particular embodiment, there are nine (9) input nodes to the classification neural network 22a and then the quantification neural networks 22b-22d. Seven of these nodes are spot signals (e.g., intensity values) from the spots 43 while two of these nodes relate to identification codes for the reagents (RID) or the fabricated flow assay cartridge (FID). Using the RID and FID as inputs produces more accurate results from the trained neural network. The RID and/or FID may be stored, for example, in bar code, QR code, serial number, or other indicia that identifies batch and reagent information. This information may be located on the flow assay cartridge 12 or it may be downloaded from a remote database. While nine such inputs to the trained neural network 22a are illustrated herein, it should be appreciated that other numbers of types of nodes may be contemplated.

### EXPERIMENTAL

### Multiplexed Sensing Membrane Fabrication and VFA Assembly

The multiplexed sensing membrane 42 contains, in one embodiment, up to eighty-one (81) spatially isolated immunoreaction or biological reaction spots 43 that are each defined by a ‘spotting condition’ which refers to the capture biomolecule such as a protein and the associated buffer dispensed onto the nitrocellulose sensing membrane 43 prior to assembly and activation. Biomolecules include molecules capable of specific binding and/or reaction with an analyte (or multiple analytes) contained in a sample. Biomolecules thus includes by way of example, proteins, antibodies, nucleic acids (e.g., DNA and RNA), aptamers, enzymes, and the like. Therefore, to design the multiplexed sensing membrane 42 for computational analysis, a custom spot-assignment algorithm was developed to generate a ‘spot map’ within the active area of the flow assay device. Based on a given grid spacing and number of spotting conditions, the assignment algorithm distributes spotting conditions such that no single spotting condition is disproportionately positioned near the center or the edge of the multiplexed sensing membrane 42. Because the vertical flow rate can vary radially across the multiplexed sensing membrane 42, leading to variations of each reaction across the sensing area of the flow assay cartridge 12, this step mitigates a potential bias on any given spotting condition. With seven spotting conditions (see FIG. 6) in a 9×9 grid format (1.3 mm periodicity), the spot-assignment algorithm produced the map of 91 spots shown in FIG. 6, which was implemented as the initial design for this study.

An automated liquid dispenser (MANTIS, Formulatrix®) was used to deposit 0.1 μL of the different protein conditions directly onto a nitrocellulose (NC) multiplexed sensing membrane 42 in the algorithmically determined pattern shown in FIG. 6. During the spotting process, up to 24 NC multiplexed sensing membranes 42 were produced on a single connected sheet, constituting one fabrication batch, and up to three batches were produced on a given day. In order to evaluate batch-to-batch variations, multiplexed sensing membranes 42 were produced over multiple fabrication batches as well as with two reagent batches (i.e., sets of reagents which had unique storage times and/or lot numbers). Each sensing membrane was therefore tagged with a corresponding fabrication batch ID (FID, e.g., 1, 2 or 3,) and reagent batch ID (RID, e.g., 1 or 2).

Following the automated spotting procedure, the NC sheets were incubated at room temperature for 4 hours after which they were submerged in 1% BSA blocking solution and allowed to incubate at room temperature for 30 min. The NC sheets were then dried in an oven at 37° C. for 10 min, after which they were cut into individual multiplexed sensing membranes 42 (1.2×1.2 cm) using a razor. The remaining paper materials contained in the VFA were produced following the methods outlined previously in Joung H-A et al., Paper-based multiplexed vertical flow assay for point-of-care testing, Lab Chip, 2019, 19, 1027-1034, which is incorporated herein by reference. All the paper materials, including the NC multiplexed sensing membranes 42 were then assembled within the top and bottom cases (52, 54) of a 3-D printed vertical flow assay cartridge 12, with foam tape holding together the paper stack (see FIG. 3).

### hsCRP Assay Procedures

Each hsCRP measurement with the flow assay cartridge 12 is performed as follows: first 5 μL of serum sample is diluted 10 times in a running buffer (3% Tween 20, 1.6% BSA in PBS) resulting in a 50 μL, sample solution. Then 200 μL, of running buffer is injected into the inlet 53 and allowed to absorb. After absorption into the paper-stack 60 (˜30 sec), 50 μL of sample solution is mixed with 50 μL of the gold-nanoparticle (Au NP) conjugate solution and the mixture is pipetted into the inlet 53 and allowed to absorb. The gold nanoparticle-C-Reactive Protein antibody (AuNP-antiCRP) conjugate is synthesized using the following protocol: (1) mix 900 μl of 40 nm AuNP solution (Ted Pella Inc., 15707-1), 100 μl 0.1M Borate buffer (pH 8.5), and 5 μl anti-CRP mouse IgG antibody (Abcam, ab8278). Incubate the mixture at 25° C. for 1 hr; (2) following the 1-hour incubation, add 100 μl of 1% BSA in PBS solution and mix by vortexing. Then incubate the mixture at 25° C. for 30 minutes; (3) transfer the mixture to the fridge and incubate at 4° C. for 2 hours; (4) centrifuge the mixture in a tube at 8000 rpm at 4° C. for 15 minutes; (5) after centrifugation, open the tube and discard the supernatant; (6) add 1 ml of 10 mM tris buffer (pH 7.4) to the microcentrifuge tube containing the AuNP-antCRP mixture and mix by vortexing; (7) repeat the centrifugation and wash steps (steps 4, 5, 6) twice to enhance the purity of the mixture; (8) add 100 μL of storage buffer (0.1 M borate buffers, pH 8.5 with 0.1% BSA and 1% sucrose) to the supernatant and mix via pipetting. The final concentration of AuNP antibody conjugates (5 OD) was confirmed by optical density measurements at 525 nm.

Lastly, after absorption of the sample solution, 400 μL of the running buffer is added to wash away the nonspecifically bound proteins and Au NPs. After a 10-minute reaction time, the flow assay cartridge 12 is then opened, and inserted into the bottom of the mobile-phone reader 10 (FIG. 1A). The mobile phone reader 10 includes a housing 28 that holds the mobile phone 14 to place the camera 16 of the mobile phone 14 along an optical path 40 that passes within the interior of the housing to the flow assay cartridge 12. The interior of the housing holds various components of the reader 10 and also ensures that ambient light does not interfere with the imaging operations described herein. The flow assay cartridge 12 in the opened state is affixed to the bottom of the housing 28 to place the multiplexed sensing membrane 42 in the optical path 40 or field of view of the camera 16 of the mobile phone 14. The interior of the housing 28 includes one or more light sources 30 such as light-emitting diodes (LEDs) that are used to illuminate the multiplexed sensing membrane 42 for imaging.

In the experiments described herein, 532 nm LEDs were used as the light sources 30. An optional diffuser 48 is used to more uniformly illuminate the multiplexed sensing membrane 42. The one or more light sources 30 may be powered by one or more batteries 34 in the housing 28 or even the mobile phone 14 itself. Driver circuitry 32 for the LEDs is also contained in the reader device 10. An external lens 38 is provided in the housing 28 to enable the camera 16 to image the entirety of the multiplexed sensing membrane 42 in focus. The housing 28 may have a mount or coupling so that the opened flow assay cartridge 12 can be temporarily secured to the housing 28 during the imaging operation as explained herein. This mobile reader 10 images the multiplexed sensing membrane 42 using the standard Android camera app (ISO: 50, shutter at 1/125, autofocused), and saves a raw image of the multiplexed sensing membrane 42 (.dng file) for subsequent processing and quantification of the CRP concentration. It should be appreciated that the mobile phone reader 10 may be manufactured to accommodate any make or model of mobile phone 14 (or other portable electronic device) and is not limited to a particular brand or model.

### Data Processing

Custom image processing software 20 was developed to automatically detect and segment the immunoreaction or biological reaction spots 43 in each mobile-phone image 50 of the activated flow assay cartridge 12 (see FIG. 9). This image processing software 20 may be run on the mobile phone device 14 itself or it may be run on a separate computing device that receives transferred image files from the mobile phone 14. This may include a local computer or a remote computer (e.g., server). After segmentation, the pixel average of each spot is calculated and subtracted by the pixel-average of a locally defined background containing BSA blocked NC membrane 42. Each background-subtracted spot signal is then normalized to the sum of all the spots on the multiplexed sensing membrane 42. The final spot signal s′_{m,p} is therefore described by,

$${s}_{m , p}^{′} = \frac{{s}_{m , p} - {b}_{m , p}}{{Σ}_{p} {Σ}_{m} ( {s}_{m , p} - {b}_{m , p} )} \qquad Eq . 1$$

where m represents the spotting condition, and the p represents the p^{th} redundancy on the VFA per condition. s_{m,p} is the pixel average of a given segmented spot, and b_{m,p} is the local background signal. The final VFA signal per condition can then be calculated as:

$${x}_{m} = \frac{1}{{P}_{m}} {Σ}_{p = 1}^{{P}_{m}} {s}_{m , p}^{′} \qquad Eq . 2$$

where Pₘ is the number of redundancies for a given spotting condition. The normalization step in Eq. (1) helps to account for cartridge-to-cartridge variations borne out of pipetting errors, fabrication tolerances, as well as operational variances.

### Clinical Testing

Remnant human serum samples were procured (under UCLA IRB #19-000172) for hsCRP testing using the system 2. Each clinical sample was previously measured within the standard clinical workflow as part of the UCLA Health System using the CardioPhase hsCRP Flex® reagent cartridge (Cat. No. K7046, Siemens) and Dimension Vista System (Siemens). In total, 85 clinical samples were measured in triplicate with the flow assay cartridge 12. All but one sample was within the standard hsCRP range of 0 to 10 mg/L, with the outlier having a concentration of 83.6 mg/L. In addition to testing these clinical samples, nine CRP-free serum samples (Fitzgerald Industries International, 90R-100) were measured as well as nine artificial samples created by spiking 200, 500, and 1000 mg/L CRP into CRP-free serum samples. These artificial samples were tested to simulate serum samples from patients undergoing acute inflammatory events. Though relatively rare in the context of hsCRP testing, such high concentration samples can be falsely reported as having a low CRP concentration due to the hook-effect. Therefore, these samples were included to test if the system 2 could avoid such false reporting. Among different batches of 273 fabricated flow assay cartridges 12, one test was removed from the data-set due to a fabrication error.

### Computational VFA Cartridge Analysis

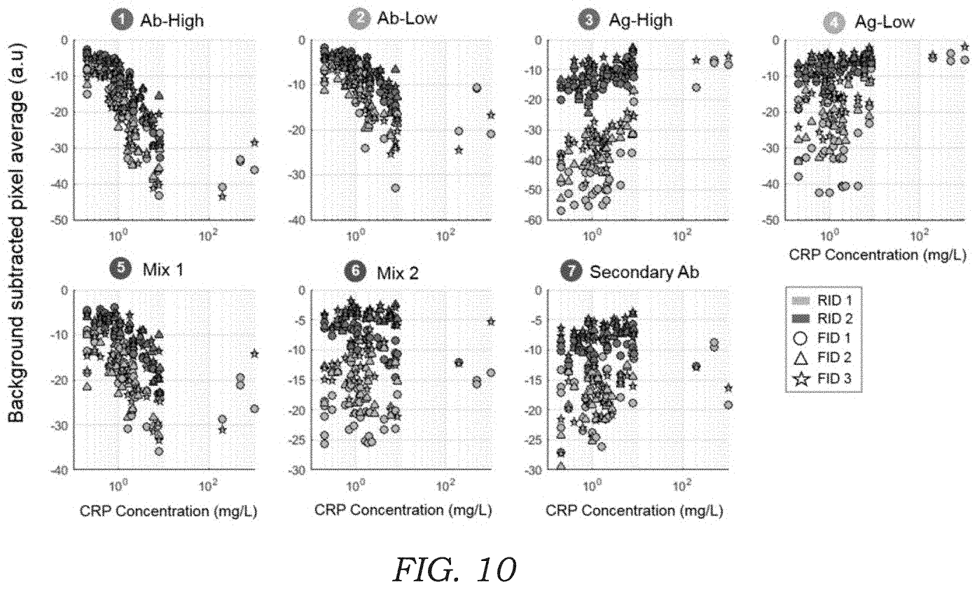

After the clinical study was completed the image data from the activated flow assay cartridges 12 were partitioned into a training set (Nₜᵣₐᵢₙ=209) and testing set (Nₜₑₛₜ=57). This data partition was structured to ensure that the testing samples would be distributed linearly over the hsCRP range, and that samples were pulled proportionally from the different fabrication batches within each cardiovascular risk stratification group. The raw background-subtracted pixel average values are shown in FIG. 10 for the seven different conditions (RID 1, RID 2, FID 1, FID 2, FID 3 data shown).

### Model and Cost Function Selection

The training set was analyzed via a k-fold cross-validation (k=5) to determine the optimal learning algorithm for quantification of CRP concentration from the inputs X_{IN}. Different fully connected networks were evaluated through a random hyper-parameter search, where the number of nodes, layers, regularization, dropout, batch-size, and cost-function were each randomly selected from a user-constrained list. A tiered neural network 22 architecture (FIG. 7) with a cost function of mean-squared logarithmic error (MSLE) yielded the best performance over the random iterations of the cross-validation. As an alternative, a single neural network with multiple hidden layers, in contrast to the tiered structure, could also be used in providing an accurate and generalizable model.

### Optimization of VFA Spots and Conditions Using Machine Learning

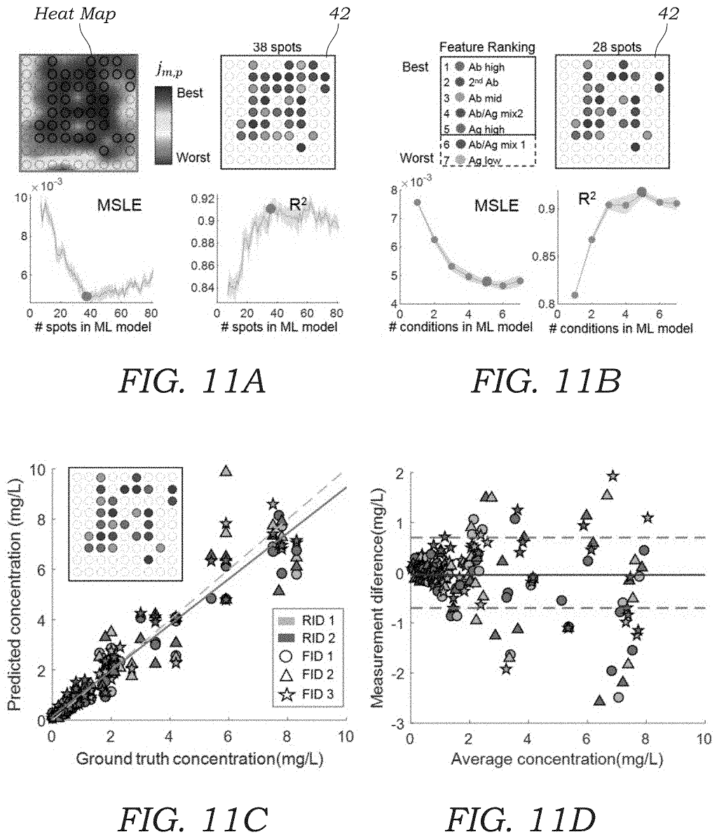

Machine learning-based optimization and feature selection of the flow assay cartridge 12 system 2 was performed in two distinct steps: spatial spot selection and condition selection, illustrated in FIGS. 11A and FIG. 11B, respectively. For the spot selection process, a cost function, j_{m,p} was defined per sensing spot to represent the normalized distance from the mean of like-spots (i.e. spots that share the same condition) averaged over the samples in the training set,

$${j}_{m , p} = {\sum }_{n = 1}^{{N}_{Train}} \frac{\vert {s}_{m , n , p}^{′} - {\overline{s}}_{m , n}^{′} \vert}{{\overline{s}}_{m , n}^{′}} \qquad Eq . 3$$

where s′_{m,n,p} is defined in Eq. 1 with the added index n indicating the n^{th} sample in the training set. s̅′̅_{m,n} is the spot signal averaged over each condition within a single test,

$$i . e . {\overline{s}}_{m , n}^{′} = \frac{1}{{P}_{m}} {\sum }_{p = 1}^{{P}_{m}} {s}_{m , n , p}^{′} .$$

The heat map in FIG. 11A, which is interpolated from a 9×9 matrix of the cost function defined at each spot of the flow assay cartridge 12, visualizes the statistically robust active areas of the multiplexed sensing membrane 42. To select a subset of spots 43 from the 9×9 grid configuration, a k-fold (k=5) cross-validation was performed. The cross validation was performed over 75 iterations where the input to the neural network, X_{IN}, was defined by incrementally smaller subsets of the original 81 spots for each iteration. The spot 43 with the maximum cost j_{m,p} was eliminated at each iteration, resulting in the last iteration containing a subset of seven (7) spots, each corresponding to a different condition. The MSLE value from the cross validation was then plotted for every iteration to visualize the trade-off between the number of spots and the error of the network inference (FIG. 11A). Due to the random training process of the neural network, there is noise associated with this curve, however a clear performance benefit can be seen after the elimination of the first 30 to 40 spots corresponding to the highest j_{m,p}. It is also clear that further reducing the number of spots results in substantial increase in quantification error. Therefore, the approximate minimum of the MSLE curve was used to define a subset of thirty-eight spots 43 for subsequent analysis.

After this initial spot selection (FIG. 11A), this subset of thirty-eight spots 43 was further subject to a condition selection step to further optimize the performance of the system 2 for hsCRP. This second phase of the feature selection aims to select the most robust sensing channels as defined by the unique chemistry attributed to the different spotting conditions. To this end, a second iterative k-fold (k=5) cross-validation analysis was performed, eliminating one spotting condition each iteration and tracking the cross-validation error as a result of each elimination. This process was repeated for incrementally smaller subsets of conditions defined by the minimum MSLE result from the previous iteration. Resulting from this analysis, FIG. 11B reports the MSLE and coefficient of determination as function of the number of spotting conditions, suggesting that eliminating the Ab/Ag Mix 1 and Ag-low condition can lead to slightly better or equivalent performance when compared to the inclusion of all the original spotting conditions (e.g., five total spotting conditions).

Taken together, this machine learning-based optimization of the VFA leads to the statistical selection of the best combination of spots and conditions (FIG. 11C inset) that can computationally determine the analyte concentration. The cross-validation results, compared to the gold standard hsCRP measurements, are also reported in FIG. 11C and FIG. 11D. Here, the inputs to the neural network, X_{IN}, are defined by the optimal spot configuration as determined by the spot and condition selection (see FIG. 11C inset), and also include two additional integer features which correspond to the reagent ID (RID∈{0, 1}) and the fabrication batch ID, (FID∈{8 1, 2, 3}).

After this feature selection and cross-validation analysis reported in FIGS. 11A-11D, the final CRP quantification algorithm was trained using the entire training set (Nₜᵣₐᵢₙ=209) and the optimal spot configuration (FIG. 11C). In addition to the CRP quantification algorithm, a second classification algorithm was trained to identify the CRP samples representing an acute inflammation event, with a CRP concentration threshold of >10 mg/L (\hat{N}ₜᵣₐᵢₙ=6, \hat{N}ₜₑₛₜ=6) (FIG. 7).

### Validation of Computational VFA Performance for CRP Measurements

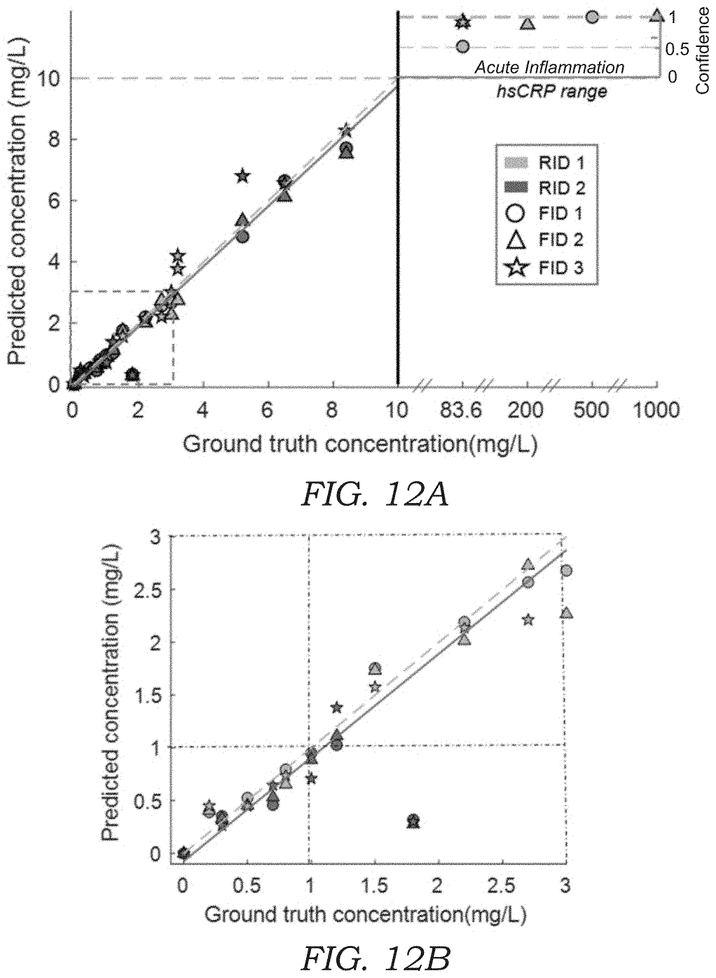

The results from the blind testing set (Nₜₑₛₜ=57) obtained with the flow assay cartridges 12 correlated well to the quantification results of the gold-standard hsCRP Flex cartridge run on the Dimension Vista System (see FIGS. 12A and 12B). These samples were analyzed using only the pixel information contained within the computationally determined subset of 28 spots and 5 conditions (FIG. 12A). The xₘ signals (Eq. 2) along with the FID and RID of each test sample were first classified by an initial neural network 22a to determine if the test was in the hsCRP range (<10 mg/L) or the acute inflammation range (>10 mg/L). The system 2 achieved 100% classification accuracy, and correctly classified 6 samples as acute and the rest (51 samples) as in the hsCRP range. The samples classified in the hsCRP range were then routed to a quantification neural network 22b-d, whereas the acute samples were simply reported as acute along with a confidence score, as summarized in FIG. 8.

The quantification accuracy of the hsCRP samples using the system 2 was characterized by a direct comparison to the gold-standard values (FIGS. 12A-12B). With 51 tests quantified in the hsCRP range, the R² value was found to be 0.95, with a slope and intercept of the linear best-fit line being 0.98 and 0.074 respectively. The overall average CV of the blind testing data was found to be 11.2% with the average CV for the low-risk, intermediate-risk, and high-risk stratified samples quantified as 11.5%, 10.1%, and 12.2%, respectively. As a reference point, the FDA review criteria for hsCRP testing state an acceptance criterion of ≤20% overall CV, with a specific CV of ≤10% for samples in the low risk category (i.e., <1 mg/L).

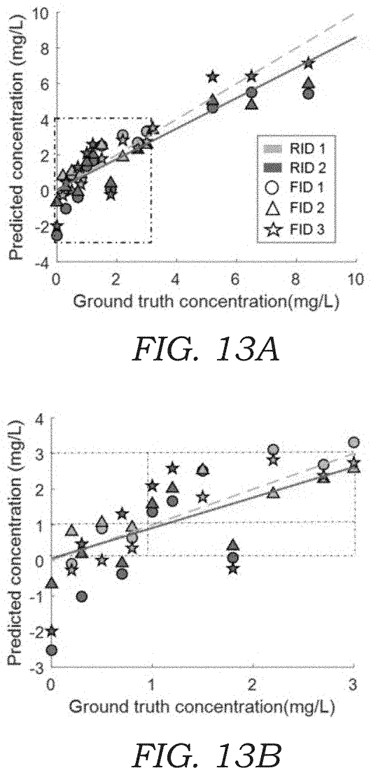

The flow assay cartridge 12-based hsCRP test benefits from machine learning in several ways. First, using neural networks to infer concentration from the highly multiplexed sensing channels greatly improves the quantification accuracy when compared to, for example, a standard multi-variable regression (see FIGS. 13A and 13B and Table 2 below).

TABLE 2

| | Neural Network | Linear Regression |
|---|---|---|
| % CV | 11.2 | 47.7 |
| % CV Low | 11.5 | 101.0 |
| % CV Int. | 10.1 | 17.7 |
| % CV High | 12.1 | 11.9 |
| R² | 0.95 | 0.79 |

Deep learning algorithms such as the fully-connected network architecture used herein, contain a much larger number of learned/trained coefficients along with multiple layers of linear operations and non-linear activation functions when compared to standard linear regression models. These added degrees of freedom enable neural networks to converge to robust models which can learn non-obvious patterns from a confounding set of variables, making them a powerful computational tool for assay interpretation and calibration. However, one concern with deep learning approaches is the possibility of overfitting to the given training set, especially in the instance of limited data. To mitigate this issue, regularization terms were incorporated in the hyper-parameter search (both L2 regularization and dropout), and found via cross-validation that the lowest error model employed the maximum degree of dropout regularization (i.e., 50%). However, it was observed that better quantification results in the blindly tested samples when compared to the cross-validation analysis, suggesting that the model appropriately generalized over the operational range of the hsCRP flow assay cartridge-based test disclosed herein.

Secondly, by incorporating fabrication information using RID and FID input features, the neural network 22 was able to learn from batch-specific patterns and signals. This resulted in a 12.9% reduction in the blindly tested MSLE when compared to the performance of a network trained without these fabrication batch input features. Similarly, incorporating the fabrication information reduced the overall CV from 16.64% to 11.2% and increased R² value from 0.92 to 0.95. It is important to note that these flow assay cartridge tests (N=273) were fabricated without the use of industry-grade production equipment such as humidity and temperature-controlled chambers, and in addition, several fabrication steps involved manual assembly. Taken together, these simple input features can benefit the performance and quality assurance of future computational POC tests following the methodology described herein. For example, the fabrication information could be included for each test in the form of a Quick Response (QR) code, bar code, serial number, or other indicia or could alternatively be logged into a GUI by the user before the measurement data are sent to the quantification network (running on a local or remote computer).

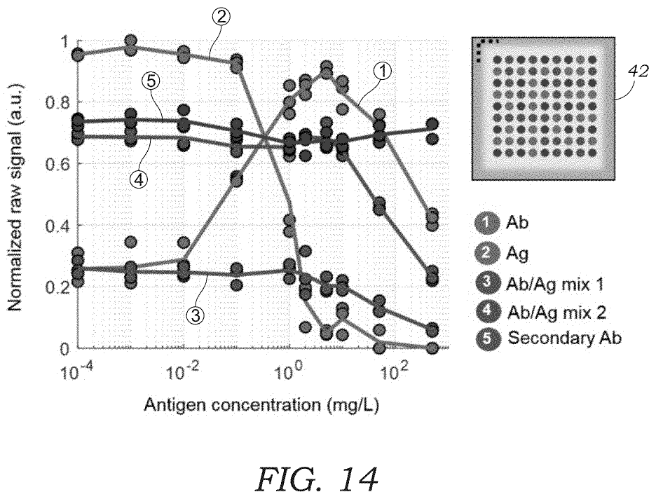

Another benefit of the system 2 is the mitigation of false sensor response due to the hook effect. The flow assay cartridge 12 format importantly enables rapid computational analysis of highly multiplexed immunoreaction or biological reaction spots 43 with minimal cross talk or interference among spots 43, which is inevitable for the case of standard lateral flow assays or RDTs. The multiplexed information reported by the different spotting conditions therefore allows for unique combinatorial signals to be generated over a large dynamic range (see FIG. 12A). The hook effect is seen in the raw sensor data, exhibited by the capture antibody (Ab) condition (see FIG. 10 and FIG. 14), illustrating how this condition alone can lead to false reporting of high analyte concentrations, i.e. in the case of acute inflammation. Therefore, without the incorporation of the monotonically responsive CRP antigen (Ag) spotting condition as one of the multiplexed channels in the flow assay cartridge 12, high-concentration CRP samples can be falsely reported as low concentration due to the hook effect. This conclusion would still be true even if another neural network was trained that used a limited number of conditions as input; for example, by re-training the classification network using only the Ab and Secondary Ab spotting conditions as inputs, it was found that the 83.6, 200, and 1000 mg/L samples are falsely reported as having CRP concentrations of 7.81, 7.34, and 3.84 mg/L respectively. In the case of analyzing only the Ab channel, all of the high-concentration CRP samples would have been falsely reported as having concentrations below 10 mg/L. These results highlight the importance of multiplexed sensing in the system 2 to mitigate the limitations induced by the hook effect in order to algorithmically enhance the dynamic range.

### Computational Sensing for Assay Development

Computational sensing broadly refers to the joint design and optimization of sensing hardware and software, and as implemented herein, provides a framework for data-driven assay development where the diagnostic or quantification algorithm informs the multiplexed cartridge design and vice versa. As detailed herein, the computational sensing approach begins with the selection of a neural network architecture and associated cost function. This first step is paramount to the design of the flow assay cartridge 12 (and more specifically the multiplexed sensing membrane 42), as it defines the model and error metric with which the subsequent feature selection is performed. The determination of the cost function therefore poses an interesting question for future diagnostic tests: because the selection of the cost function defines the training of a neural network, one needs to know the most clinically appropriate error functions with which one should design the system 2. For example, in the case of cardiovascular risk stratification with the hsCRP test, an error of ±0.1 mg/L is more problematic for samples that are in the range of the clinically defined cutoffs (i.e. 1 and 3 mg/L) when compared to samples with relatively higher CRP concentrations, such as 8 mg/L. Therefore, a traditional cost function for regression such as the mean-squared-error may not be as appropriate as the mean-squared-logarithmic-error or mean-absolute-percentage error, which take into account the relative ground-truth concentration for each error calculation. Therefore, special consideration must be given to the cost functions employed, and custom cost functions defined jointly by physicians/clinicians and engineers should be considered.

Feature selection and machine learning based optimization can similarly be used to inform the multiplexed sensing membrane 42 design. POC sensors can especially benefit from feature selection to circumvent noise borne out of their low-cost materials (such as paper used in the flow assay cartridge 12) and operational variations. For example, the heat-map in FIG. 11A very well reveals how the immunoreaction spots closest to the edges of the multiplexed sensing membrane 42 contain the most variation in their normalized signals. This most likely results from the position-dependent vertical flow variations inherent in the inexpensive flow assay cartridge 12 format, which uses paper materials totaling <$0.2 per CRP test (Table 1). These areas can therefore be avoided in future iterations, saving reagent costs and fabrication time, while also preserving robust sensing channels. Furthermore, identifying these areas of statistical variation can also inform the fabrication process. For example, FIG. 11A also shows in the heat map that the top edge of the multiplexed sensing membrane 42 as statistically more robust than the bottom and sides of the multiplexed sensing membrane 42. Therefore, this spot selection analysis indicates a unidirectional fabrication bias in the lateral alignment of the sensing membrane within the porous layer 60 stack, which can be addressed in future iterations of the batch fabrication process.

Complementing the spot 43 selection, the statistical condition selection process investigates the efficacy of the sensing channels and the unique immunoreactions defined by their spotting condition. Inherent complexities of the underlying chemistry such as the stochastic arrangement of the capture proteins within the porous NC membrane 42, as well as the effects of steric hindrance, pH, humidity, and temperature can obscure intuition behind the selection of spotting conditions for a given sensing application. Therefore, computational sensing systems can benefit from data-driven selection of sensing channels. For example, FIG. 11B shows that the quantification performance improves slightly upon the out-right elimination of the Ab/Ag Mix 1 and Ag-low conditions. This suggests that their signal response is redundant or less stable when compared to the other conditions, and is confirmed by the poor repeatability of the Ag signal between the reagent and fabrication batches (see FIG. 10). Such a feature selection procedure in a highly multiplexed format like the vertical flow assay cartridge 12 could therefore be used to computationally screen spotting conditions from a large number of differing capture chemistries including, but not limited to, different structures of capture antibodies/antigens (i.e., polyclonal vs. monoclonal) as well as varying buffer conditions and reagent concentrations. Conditions which do not empirically benefit sensor performance can be replaced by new conditions in another iteration of the development phase, or be replaced by additional redundancies of effective conditions in order to benefit from signal averaging.

Additionally, this statistical feature selection and optimization process can inform cost-performance trade-offs to help design the most robust and cost-effective implementations of POC assays. For example, the reagent cost for the immunoreaction spots contained in the hsCRP VFA test is reduced by 62%, from $2.61 to $0.97 per test, by implementing only the computationally selected chemistries. Additionally, certain spotting conditions might have an optimal capture protein concentration due to steric hindrance effects or higher degrees of nonspecific binding. Therefore, in a computational flow assay device, reagent costs can be significantly reduced without sacrificing assay performance by employing these statistically optimized capture-protein concentrations. One should also note that these reagent costs per test would be significantly reduced under large scale manufacturing, benefiting from economies of scale, which is expected to bring the total cost per test (including all the materials and reagents) to <$0.5.

Taken together, a computational POC flow assay cartridge 12 for hsCRP testing over a large dynamic range has been demonstrated. The multiplexed sensing membrane 42 contained in the system 2 was jointly developed with a quantification algorithm based on a fully-connected neural network architecture. First, a training data-set was formed by measuring human serum samples with the VFA. Then, through cross-validation of the training set, the most robust subset of sensing channels was selected from the multiplexed sensing membrane 42 and used to train a CRP quantification network 22. The network 22 was then blindly tested with additional clinical samples and compared to the gold standard CRP measurements, showing very good agreement in terms of quantification accuracy and precision. Additionally, the multiplexed channels and computational analysis helped overcome limitations to the operational range of the CRP test borne out of the hook-effect. The results demonstrate how a computational sensing framework and multiplexed flow assay design can be used to engineer robust and cost-effective POC tests that have the potential to democratize diagnostics and expand access to care.

While embodiments of the present invention have been shown and described, various modifications may be made without departing from the scope of the present invention. The invention, therefore, should not be limited except to the following claims and their equivalents.

1. A method of detecting the presence of and/or quantifying the amount or concentration of one or more analytes in a sample using a flow assay cartridge comprising a plurality of absorption layers including a multiplexed sensing membrane, the method comprising:
providing the flow assay cartridge comprising the multiplexed sensing membrane having a plurality of immunoreaction or biological reaction spots of varying conditions spatially arranged across the surface of the membrane defining a spot map, wherein the spot map comprises a pre-defined spot map of spot locations and spot conditions established for the one or more analytes;
loading a sample and reagent mixture into or onto the flow assay cartridge;
imaging the multiplexed sensing membrane with a reader device configured to illuminate and obtain one or more images of the multiplexed sensing membrane;
subjecting the one or more images to image processing with image processing software to obtain normalized pixel intensity values of the plurality of immunoreaction or biological reaction spots; and
inputting the normalized pixel intensity values to one or more trained neural networks configured to generate one or more outputs that: (i) quantify the amount or concentration of the one or more analytes in the sample, and/or (ii) indicate the presence of the one or more analytes in the sample, and/or (iii) determine a diagnostic decision or classification of the sample.

2. The method of claim 1, wherein the pre-defined spot map is determined by machine learning-based optimization.

3. The method of claim 1, wherein the amount or concentration of the one or more analytes comprises one of a qualitative output or a quantitative output.

4. (canceled)

5. The method of claim 1, wherein the one or more analytes comprises C-Reactive Protein (CRP).

6. The method of claim 1, wherein the reader device further comprises a portable electronic device having a camera configured to obtain one or more images of the multiplexed sensing membrane.

7. The method of claim 6, wherein the reader device is configured to obtain a plurality of images of the multiplexed sensing membrane to increase detection sensitivity.

8. The method of claim 6, wherein the one or more trained neural networks is executed on the portable electronic device.

9. The method of claim 6, wherein the portable electronic device comprises a mobile phone, tablet PC, laptop, camera, or microcomputer.

10. The method of claim 6, wherein the one or more images obtained by the portable electronic device are transferred to a computing device that executes image processing software and the one or more trained neural networks.

11. The method of claim 1, wherein the one or more images obtained by the reader device are subject to image processing using an on-board computing device configured to execute image processing software and the one or more trained neural networks.

12. The method of claim 1, wherein the one or more trained neural networks is executed locally on the reader device or on a personal computer, laptop, tablet, server, or other computing device.

13. The method of claim 1, wherein the flow assay cartridge is associated with bar code, QR code, serial number, or other indicia that identifies batch and reagent information.

14. The method of claim 1, wherein the sample and reagent mixture comprises gold nanoparticles conjugated to a molecular probe, an antibody, a plurality of antibodies or probes.

15. The method of claim 1, wherein the sample and reagent mixture comprises a fluorescent reporter conjugated to a molecular probe, an antibody, a plurality of antibodies or probes.

16. The method of claim 1, wherein the sample and reagent mixture comprises quantum dots or fluorescent tags conjugated to a molecular probe, an antibody, a plurality of antibodies or probes.

17. The method of claim 1, wherein the flow assay cartridge comprises one or more paper layers disposed therein and containing, in a dry state, a tag or reporter conjugated to a molecular probe, an antibody, a plurality of antibodies or probes.

18. The method of claim 17, wherein the tag or reporter comprises gold nanoparticles conjugated to a molecular probe, an antibody, a plurality of antibodies or probes.

19. The method of claim 17, wherein the tag or reporter comprises a fluorescent reporter conjugated to a molecular probe, an antibody, a plurality of antibodies or probes.

20. (canceled)

21. The method of claim 1, wherein prior to loading of the sample and reagent mixture, the flow assay cartridge is loaded with a buffer solution.

22. The method of claim 21, wherein a second buffer solution is loaded into or onto the flow assay cartridge after loading of the sample and reagent mixture.

23. The method of claim 1, wherein the immunoreaction or biological reaction spots comprises one or more of a protein, antigen, antibody, nucleic acid, aptamer, and enzyme.

24. The method of claim 1, wherein the flow assay cartridge is a vertical flow assay cartridge or a lateral flow assay cartridge.

25-44. (canceled)
